(19) United States
(12) Patent Application Publication
(10) Pub. No.: US 2026/0152714 A1
(43) Pub. Date: Jun. 4, 2026

(54) LYOPHILIZING WATER SOLUBLE MICROBIAL POWDER

(71) Applicants: Steven Shoup, New Plymouth, ID (US); Barry Roberts, Sandy Springs, GA (US)

(72) Inventors: Steven Shoup, New Plymouth, ID (US); Barry Roberts, Sandy Springs, GA (US)

(21) Appl. No.: 19/459,084

(22) Filed: Jan. 26, 2026

Related U.S. Application Data
(62) Division of application No. 18/464,489, filed on Sep. 11, 2023.

(51) Int. Cl. C12N 1/04; A23B 2/92; A23L 29/00; C12M 1/00; C12M 1/06; C12M 1/34

(52) U.S. Cl. CPC C12N 1/04; A23B 2/92; A23L 29/065; C12M 27/02; C12M 41/26; C12M 47/00

(57) ABSTRACT

Compositions and methods are provided for lyophilizing microbes suspended in a medium. Specifically, the medium, comprised of a food source and a lyophilization buffer, is 100 percent soluble in water after the lyophilizing.

### CROSS REFERENCE TO RELATED APPLICATION

This application claims priority to U.S. Provisional Application No. 63/509,292, filed Jun. 21, 2023, which is incorporated herein by reference in its entirety. In addition, this application is a divisional application which claims priority to U.S. Utility application Ser. No. 18/464,489, filed Sep. 11, 2023,

### BACKGROUND OF THE INVENTION

Global food insecurity is a chronic issue that is likely to be aggravated with increasing population growth and by the deceasing availability of arable land. Over the last 50 years, fertilizer and pesticides have been used to artificially stimulate plant growth and suppress harmful pathogens that can destroy crops and reduce yields. However, intensive use of agrochemicals has led to an increasing dependence on artificial nutrients, an increase in trapped nutrients in challenged soils, rampant expansion of crop-destroying soil-borne diseases, pathogenic organisms and destructive parasitic nematodes. The net result has been significant destruction of natural and vital soil microbial activity.

Decades of using toxic, chemically engineered fertilizers, pesticides, and soil amendments have severely damaged and sterilized agricultural soil systems and contaminated surface waters and aquifers. The accumulation of these chemicals in our food and water supply has contributed to a dramatic increase in cancers, diseases, and cognitive issues in humans and wildlife. Consequently, growers are seeking cost effective, organic alternatives to chemical-based fertilizers, pesticides, and soil amendments.

All of this has resulted in a material reduction of crop yields and nutrient quality and a need for highly effective natural soil remediation and restorative solutions. In an effort to support a pathway to longer-term sustainable and regenerative agricultural practices, companies have developed all-natural, organic and powerful natural fertility soil solutions.

Plant growth promoting microorganisms have been demonstrated to enhance plant productivity and increase crop yields. Soil amendments have been formulated that contain microbes. The rhizosphere is the soil region that subject to the influence of plant roots. In naturally fertile soils, the rhizosphere can be home to a rich diversity of microorganisms, many of which benefit plants by suppressing pathogenic invasions and helping the plants to acquire vital nutrients from the soil. These microbes create a defensive barrier around the plant roots significantly reducing soilborne pathogens and plant parasitic nematodes. As well, microorganisms that live on or near the plant root systems can significantly improve nutrient absorption and water retention of the soil. In addition, the microbes activate soil nutrients and releases bound natural fertilizers to produce more nutritious crops and enhance plant growth.

For maximum effectiveness, it is important to have a large concentration of a broad spectrum of the microbial isolates. For example in the agriculture industry, rhizobacteria can suppress pathogen ingress into the plant roots and are essential for biological nitrogen fixation. Actinomycetes produce powerful antibiotic compounds and eradicate a large array of soil borne pathogens and nematodes. Other bacterial isolates can accelerate the microbial processes that augment the availability of nutrients in a form easy to assimilate by plants. Similarly, fungi are organisms that can secrete growth promoting hormones. In addition, saprophytic fungi can produce secondary metabolites that are active in suppressing soil borne pathogens and plant parasitic nematodes. Endomycorrhizae live in the root tissue and are powerful growth stimulators. Ectomycorrhizae form a sheath around plant roots preventing nematodes and pathogen penetration as well as promote nutrient absorption. In general, soil amendments with higher microbial abundance and diversity have a greater chance of containing microbes that perform a particular beneficial function under a range of conditions.

However, growing multiple different microbes together or having them coexist can be problematic. Many microbes are antagonistic to each other or have faster growth rates at certain conditions monopolizing essential nutrients. Thus, incorporating wide spectrum of microbes is simultaneously as challenging as it is desirable.

Naturally, the greater the concentration of the beneficial microbes in the soil amendment, the less of the soil amendment is required and thus produces a more economically viable product. In order to create a high concentration of microbes in a soil amendment, it is important to have a food source that is extremely nutritious for the broad array different microbes. The media generally needs to contain sources of carbon, nitrogen and vitamins. Molasses is a widely utilizable carbon source for microbes and is the most commonly used growth media. Nitrogen sources can often include peptone, yeast extract, and malt extract. Again, the problem exists on how to ensure all the different classes of microbes can grow without monopolizing the food source required by the other microbes.

The industry typically brews the microbes in large batches with molasses. Molasses is an extremely processed product but is relatively inexpensive. It has lots of its sugar content removed during its first boiling and creation of a first syrup. Because of its extreme processing, molasses typically has material that the microbes do not consume and do not dissolve in water. In addition, molasses generally needs additional vitamins and amino acids to be added to grow some of the beneficial microbes. Often, these additions are not water soluble.

A commercial soil amendment will optimally contain a high concentration of a wide spectrum of beneficial microbes that is easily shipped and stored. Consequently, a lyophilized powder is preferred. As well as dramatically increasing shelf life, freeze-drying the soil amendment reduces their weight and volume, helping cut down on shipping costs and reduces environmental impact. These benefits are especially advantageous when transporting the soil amendments overseas to places with limited facilities and budgets. With lyophilization, a temperature-controlled distribution chain is usually not necessary. Many microorganisms survive well when lyophilized and can be easily rehydrated and rejuvenated after prolonged periods of time in storage.

A commercial sprayer is often used to apply soil inoculants and plant fertilizers. The same agricultural sprayers can be used to apply the microbial based soil amendments. In this case, it is critical that dry soil amendment is completely water soluble to prevent a host of mechanical issues such as the sprayer and other components becoming clogged. However, it is challenging to create a lyophilized soil amendment that is water soluble.

A further problem exists when one type of microbe grows too rapidly. The brew will tend to form a film or a matty layer created by the over-abundance of a particular microbe. These matty layers or matts can be found on the top of the brew, on the bottom. or somewhere in the middle depending on the particular microbe. This matty layer creates a problem during the processing of batch into a marketable soil amendment. These matts are difficult to break down and are difficult to filter out of the brew. Consequently, these matts present an issue for creating a water-soluble product.

Thus, there is continuing need for improved biological soil amendments. The improved soil amendment needs a to contain a high microbial abundance with a diversity of classes of microbes while being water soluble. Numerous other fields can benefit from similar all-natural organic solutions.

### BRIEF SUMMARY OF THE INVENTION

The present invention discloses compositions and methods in various embodiments for enhancing the concentration of microbes with a diversity of classes suspended in a water-soluble powder after lyophilization.

In one embodiment, a composition comprising of lyophilized microbes is suspended in a medium that is 100 percent soluble in water after lyophilizing at ambient temperatures and pressures. The medium is comprised of a lyophilizing buffer and the food source. Preferably, the medium also includes a pH increasing solution that is soluble in water.

In this embodiment, the food source includes at least one of amino acids and/or vitamins. The food source is comprised of a plurality of components which are all soluble in water. In addition, the lyophilizing buffer is comprised of components that are all soluble in water.

The food source preferably includes honey and a yeast source. In order to be water soluble, the honey is processed to have its wax component removed. The yeast source is a water-soluble yeast extract. The microbes grow best when the yeast source is greater than 20 percent of the food source.

In this embodiment, the lyophilizing buffer is comprised of sugar. Sugar cane is preferred to create the excipient. The sugar is then mixed with water to form a solution between 5 percent and 10 percent inclusive of sugar after adding the buffer to the microbes or the microbial solution.

In another embodiment, a method of lyophilizing microbes in a 100 percent soluble in water medium at ambient temperatures and pressures after the lyophilizing is disclosed. The medium comprises of a lyophilizing buffer and a food source.

The microbes are grown by using the food source that is completely water soluble. In addition, the food source includes at least one of amino acids and/or vitamins. The food source preferably comprises of honey and a yeast source. In order to be water soluble, the honey is processed to have its wax component removed. The yeast source is comprised of yeast extract. The yeast source is greater than 20 percent of the food source.

In this embodiment, the lyophilizing buffer is comprised of sugar cane. The sugar cane is mixed with water to form a solution between 5 percent and 10 percent inclusive of sugar after adding the buffer to the microbes or the microbial solution.

In another embodiment, a method of lyophilizing microbes in a medium is disclosed. The process includes cooking a food source that has amino acids, adding the food source to the microbes, adding a lyophilizing buffer to the microbes, lyophilizing the microbes to suspend the microbes in a medium, and powderizing the medium. The medium is 100 percent soluble at ambient temperatures and pressures after lyophilization.

In this embodiment, the powderizing of the medium includes turning the medium into a course powder by using a shaking mechanism. Using another shaker mechanism to turn the coarse powder into a fine powder. The process of powderizing the medium works best when performed at or below 75 degrees Fahrenheit and/or 33% humidity.

The lyophilizing buffer is comprised of cane sugar. The sugar cane is mixed with water to form a solution between 5 percent and 10 percent inclusive of sugar after adding the buffer to the microbes or the microbial solution.

The cooking of the food source is performed at a temperature and time sufficient to kill contaminating microbes.

### BRIEF DESCRIPTION OF THE DRAWINGS

Various embodiments are illustrated by way of examples in the figures of the accompanying drawings. Such embodiments are demonstrative and not intended to be exhaustive or exclusive embodiments of the present subject matter. The drawings are not necessarily to scale, emphasis instead being placed on illustrating the principals of the invention.

FIG. 1 is a flow chart depicting the major steps of the preferred embodiment of the present invention.

FIG. 2 depicts the step of cooking a microbial food source.

FIG. 3 depicts the step of brewing a microbial solution.

FIG. 4 depicts the lyophilizing a microbial medium.

FIG. 5 depicts the step of crumbling the microbial medium.

FIG. 6 depicts the step of milling the microbial medium.

FIG. 7 depicts the step of shaking the medium into a blended powder.

FIG. 8 depicts the step of diluting the final product for packaging.

### DETAILED DESCRIPTION OF THE INVENTION

The description that follows includes compositions, systems, methods, and apparatuses that embody various elements of the present disclosure. However, it should be understood that the described disclosure may be practiced in a variety of forms in addition to those described herein. Accordingly, the referenced drawings show, by way of illustration, specific embodiments in which the invention may be practiced. It is to be understood that other embodiments may be utilized, and structural changes may be made without departing from the scope of the claims. It is further understood that the steps described with respect to the disclosed processes may be performed in differing order and are not limited to the order presented herein.

The present invention discloses compositions and methods in various embodiments for enhancing the concentration of microbes with a diversity of classes suspended in a water-soluble powder after lyophilization. The preferred embodiments are described below.

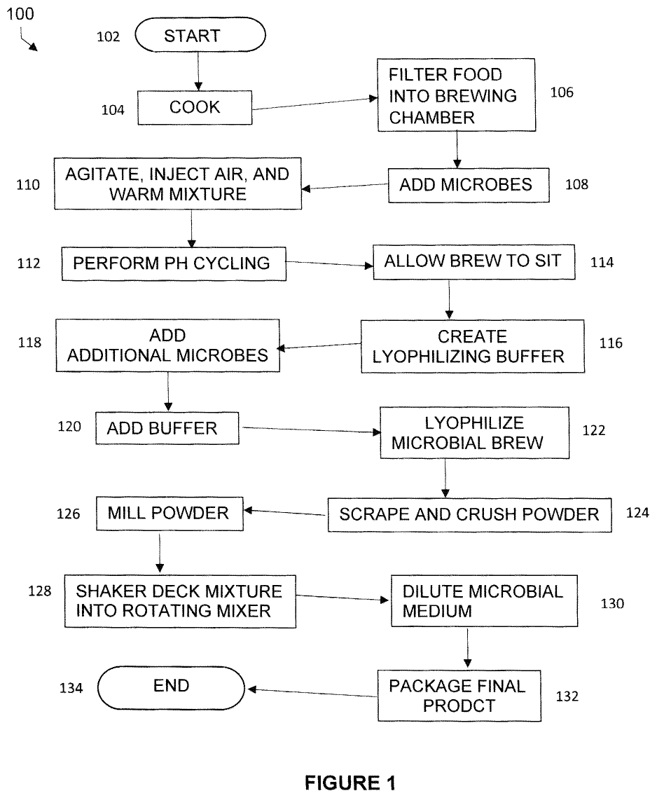

Referring to FIG. 1, illustrated is a flowchart 100 for the major steps of enhancing the concentration of microbes with a diversity of classes suspended in a water-soluble powder.

Following the start step 102, a food source is cooked as described in step 104.

In step 104, care should be used to choose foods and components that are 100% water soluble and stay in suspension to help ensure water solubility of the final product.

Honey should be considered as a food source since it contains a high concentration of sugars and essential vitamins especially the B vitamins. However, honey has low glass transition temperatures (Tg), which makes freeze drying difficult. During dehydration, honey typically becomes extremely sticky, hard and very difficult to handle as a product. Although honey does not lyophilize well, when used as a food source, the microbial count skyrockets compared to most other food sources.

Start with 50% brewer's yeast by weight and 50% processed honey by weight. The brewer's yeast selected should be 100% water soluble. Processed honey with the waxes removes is used to help ensure solubility. The industry typically uses around 10% yeast; however, a 50% yeast mixture increases the yield when combined with honey. To ensure consistency, the honey should be a blend that uses a large variety of honeys from multiple sources.

In step 106, the food source is filtered and added into a brewing chamber. Cool the food mixture to below 95 degrees. The cooked mixture is then filtered and cleaned down to at least 10 microns before going into a brewing vessel. The filtering removes most of the non-soluble material in the solution including the yeast cell wall debris. The resultant is approximately 39 to 40 liters of food to be added to the brewing vessel.

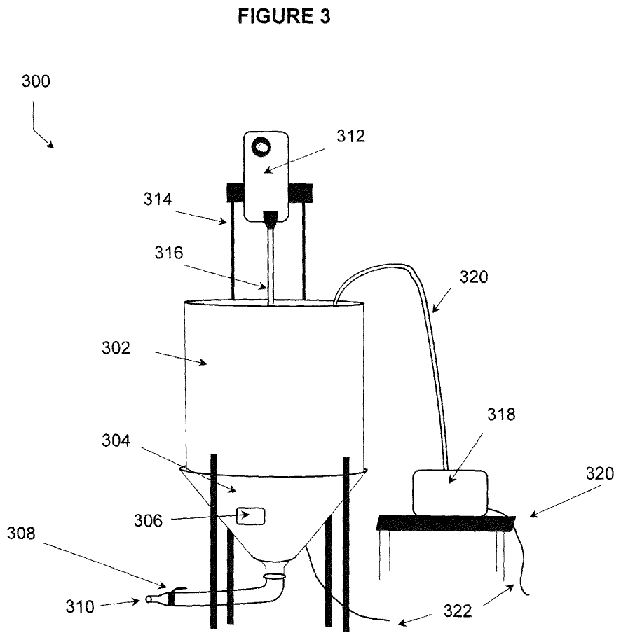

In step 108, after adding the food mixture, 8 liters of microbes from a previous batch is added to the feeding tank. It is preferred to have a broad spectrum of the microbial isolates. Rhizobacteria can suppress pathogen ingress into the plant roots and are essential for biological nitrogen fixation. Actinomycetes produce powerful antibiotic compounds and eradicate a large array of soil borne pathogens and nematodes. Other bacterial isolates can accelerate the microbial processes that augment the availability of nutrients in a form easy to assimilate by plants. Similarly, fungi are organisms that can secrete growth promoting hormones. In addition, saprophytic fungi can produce secondary metabolites that are active in suppressing soil borne pathogens and plant parasitic nematodes. Ectomycorrhizae form a sheath around plant roots preventing nematodes and pathogen penetration as well as promote nutrient absorption. By using the disclosed pH cycling, the concentration of these microbes is greatly increased and are brewed together. The step of brewing 108 of the microbial solution is depicted in reference to FIG. 3.

The step of brewing 110 of the microbial solution is depicted in reference to FIG. 3. In step 110, agitation and air injection at bottom is started after adding the microbes. Compressed and dried filtered air is injected into the feeding tank. Direct oxygen can be detrimental. A light bubble similar to a fish tank is utilized just to keep brew oxygenated. The stirring rotates at 45 to 70 rpm to keep the mixture moving and lightly agitated. External heating pads surrounding the vessel are set to keep the mixture to around 85-95 degrees.

In step 112, after adding the microbes, the resultant mixture is around 6.3 pH. In order to maximize the growth of a wide diversity of microbes with the temperature held constant, the pH needs to be managed such that all the microbes grow without any one microbe severely dominating. Accordingly, the brewing during the growing process is controlled to a pH of around 6 to a lower pH in the mid 4s. This cycle allows the different groups of microbes to multiply. Since different microbes thrive at differing pHs, cycling the pH ranges allows the microbes to grow together to feed and multiply without other microbes dominating. If the brew is allowed to sit too long in the low 4s pH, a resultant acidophilic actinomycetes bloom occurs. The cycling of the pHs allows the microbes to grow without any one particular microbe dominating and eliminates the matting layer issue.

Mined sodium carbonate that is completely water soluble is preferred to be used to alter the pH. Use 5% mixture of sodium carbonate to water. When the brew is in the mid 4s pH, add sodium carbonate while continuing to agitate. The first pH crash to mid 4s pH usually takes around 5 to 5.5 hours and will need about 2 liters of the sodium carbonate to raise the batch to around a 6 pH. The second crash to the mid 4s PH usually takes less time of 3 to 4 hours and the addition of around 3 liters to raise the pH back to around a pH of 6. The third crash may take as long as 13 hours. Typically, the total of 8.5 to 9 liters of the sodium carbonate is used for the 3 runs.

Running the brew through two pH cycles greatly increases the microbial yields of all the differing microbes. A third cycle is preferred to be performed to further increase microbial count. At the end of the pH brew cycling, the brew will be at approximately a 6 ph.

In step 114, allow the brew to sit for another 48 hours in order to maximize count. As the brew sits, the brew will creep up into 7s pH. Some of microbes will diminish if the brew hits an 8 pH.

In step 116, the lyophilizing buffer or excipient is created. In a large mix vessel, add 3200 grams of cane sugar to water until the total volume is 4 liters. Mix until the cane sugar is fully dissolved. Ideally, the mixture is concentrated to act as a buffer with the cane sugar staying suspended at room temperature. In order to make the product rise as a puff cake, an 8% mixture is used. Over a 10% concentration will start killing microbes as the sucrose is toxic to some of the microbes. Under 5%, the end product is sticky and gooey and is difficult to create a water-soluble product.

In step 118, any additional microbes are added to the microbial brew. If endo-mycorrhizae are to be added to brew, it should be added at this time. Endo-mycorrhizae may take up to 10 days to brew. Most endo-mycorrhizae are grown on plant roots and harvested by scraping or crushing the roots. Endo-mycorrhizae grown this way make the product non-water soluble. However, certain foods can allow endo-mycorrhizae to grow using the methodology described in this invention and can be water soluble.

In step 120, add the sugar solution to brew containing the microbes. Turn the agitator up and slowly add the sugar buffer solution. The lyoprotectant used in this embodiment of the invention is sucrose, which is a glucose linked via its C1 carbon to fructose. By using honey and sucrose as a dehydration buffer, the final product becomes a fluffy cake with a fine texture and excellent solubility characteristics. If there is not enough sugar, the end product is a sticky mass that clumps because of honey and resists becoming suspended in water.

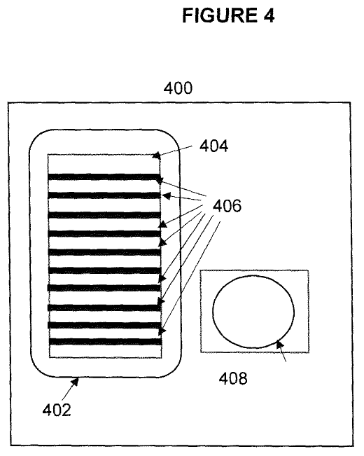

In step 122, the microbial medium is lyophilized as depicted in reference to FIG. 4. Pour the buffered solution into trays for lyophilization. In this preferred embodiment, 2100 ml is poured into a tray and then set programming. Lyophilize the solution for approximately 60 hours to 70 hours. Preserving microbes by lyophilization necessitates that the microbes are suspended in a medium that helps to maintain their viability through freezing, water removal, and subsequent storage. The ideal solution will have a component that helps to form a solid “cake” which gives body to the bacterial suspension once freeze dried.

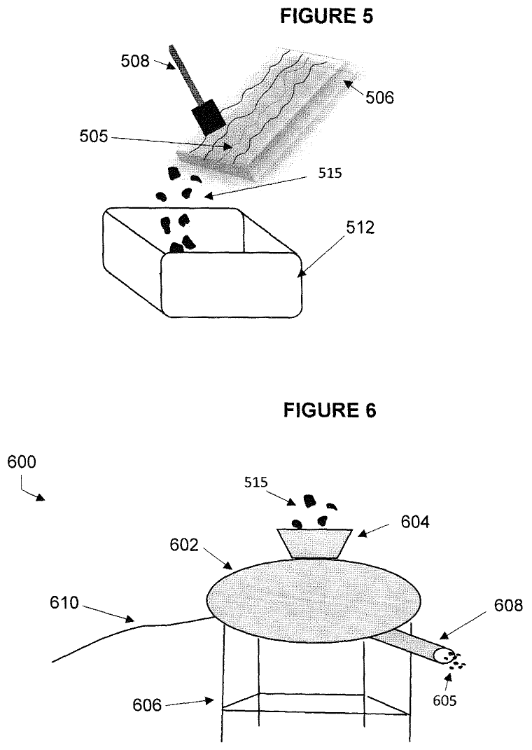

In step 124, after lyophilizing, the microbial medium is removed from the trays and placed into a tub as depicted in reference to FIG. 5. After removing the microbial medium from the lyophilizer, the environment should be controlled to be less than 75° F. and less than 33% humidity. Outside of those parameters, the medium tends to absorb water, and thus, creating a fine powder is extremely difficult. Using a mallet, gently crush the microbial medium into crumbs. In the preferred embodiment, the microbial medium has an airy cake texture and easily obtains a crumbly consistency with slight pressure.

In step 126, a brewer's grain mill crushes the microbial as depicted in reference to FIG. 6. In the preferred embodiment, the brewer's mill uses two stainless steel slow rollers gapped at approximately one millimeter. The microbial medium leaves the brewer's mill as a coarse powder.

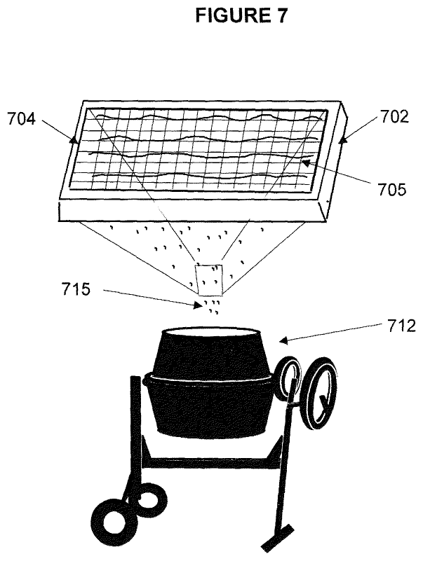

In step 128, the microbial is mixed with dextrose as depicted in reference to FIG. 7. Blend the microbial powder with dextrose with 2 to 3 parts bulking agent (such as dextrose, sucrose, or other fine water soluble particulate bulking agents) to 1 part microbial powder. Preferably, use a fine dextrose product without particulates that is clean and does not fall out in a water solution. Shake the coarse microbial powder and the dextrose on a shaker screen using a 20-mesh to 200-mesh screen to create a blended powder. The blended powder enters into a horizontal cement mixer to ensure the blended powder stays moving.

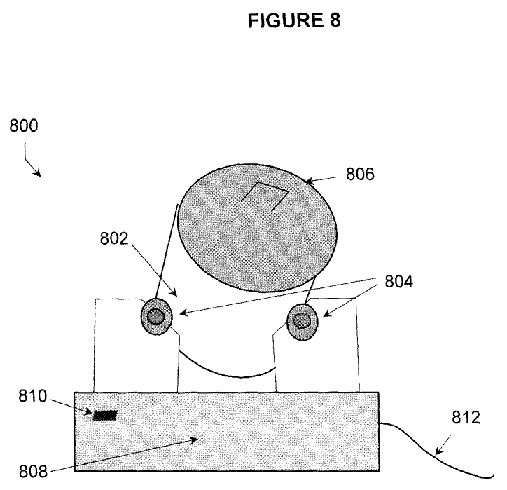

In step 130, the microbial blend is further diluted with dextrose. A tumbler, as depicted in reference to FIG. 8, is used to dilute the end product until achieving the final desired dilution such as 5 parts dextrose to 1 part fine microbial powder.

In step 132, the diluted microbial powder is packaged. After dilution, seal the powder in an air-tight package. After packaging, the product is ready for sale, which ends the process as shown in step 134.

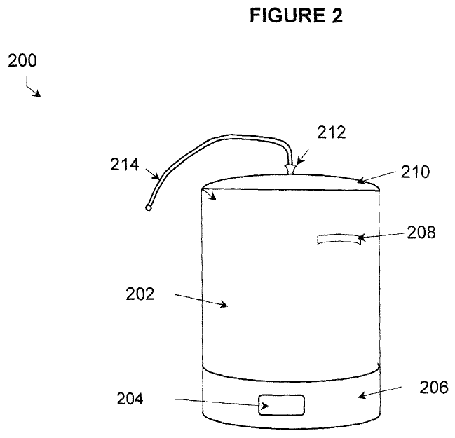

Referring to FIG. 2, the step of cooking 104 the microbial food source is depicted. In the preferred embodiment, 1000 grams of honey is mixed with 1000 grams of brewer's yeast in a commercial cooker 200. The commercial cooker 200 has a cooking chamber 202 with a handle 208, a lid 210, a relief valve 212 with attached tubing 214, and a heating element casing 206 with a heater control panel 204. Water is added until there is 42 liters of mixture is in the cooker. The mixture is cooked at a simmer for 20 minutes. This cooking kills any live unwanted bacteria in the water and breaks open the cells of the yeast to create a yeast extract. The cooking temperature should not be hot enough to denature the proteins.

Referring to FIG. 3, depicted is the step of brewing 108 of the microbial solution in a brewing vessel assembly 300. The brewing vessel 302 is an insulated stainless steel cone bottom tank. A nozzle valve 308 at the bottom of the brewing assembly 302 enables the solution to be withdrawn after brewing via the exit nozzle 310.

Agitation is provided by an agitator rotation rod 316 supported by the agitator support stand 314. The agitator controller 312 rotates the agitator rotation rod 316 speed at 45 to 70 rpm to keep the mixture moving and lightly agitated.

Air injection at bottom of the brewing vessel 302 is started after adding the microbes. Compressed and dried air is run through medical grade air filtration system 318 before injection into the feeding tank 302 by an air tube 320. Direct oxygen can be detrimental. A light bubble similar to a fish tank is utilized just to keep brew oxygenated. Power is supplied to the air filtration system 318 by a power cord 322.

A heating control panel 306 governs the external heating pad 304 that surround the bottom conical section of the vessel 302. The heating pad 304 is set to 90° F. warm, which should keep the mixture to around 85-95 degrees. Power to the vessel assembly 300 is supplied via the power cords 322.

In order to maximize the growth of a wide diversity of microbes with the temperature held constant, the pH needs to be managed such that all the microbes grow without any one microbe severely dominating. Running the brew through two pH cycles greatly increases the microbial yields of all the differing microbes. A third cycle is preferred to be performed to further increase microbial count.

Referring to FIG. 4, depicted is the step of the lyophilizing 122 the microbial medium. The microbial medium is poured into trays 406 and placed on the shelves 404 in the vacuum chamber 402 of the lyophilizer 400 for dehydration. A motor (not shown) is in place to ensure the vacuum chamber 402 is vacuum-tight when closed. The condenser 408 consists of refrigerated coils or plates. During the drying process, ice moves from the frozen product to the condenser by sublimation.

Referring to FIG. 5, depicted is the step of transferring 124 the post lyophilized microbial medium 505 from the lyophilization tray 406 into a container 512. The microbial medium 505 ideally has an airy cake texture that easily obtains a crumbly consistency with slight pressure. In this embodiment, a spatula 508 is used to scrape the post lyophilized medium 505 from the tray 506. Gently crush the post microbial medium 505 into crumbs 515.

Referring to FIG. 6, the step of pouring 126 the crumbs 515 into a brewer's grain mill 600 is depicted. The brewer's mill 600 is supported by a stand 606 and powered by a power cord 610. The microbial medium crumbs 515 are poured into the mill's funnel 604. In this embodiment, the brewer's mill 600 uses two stainless steel slow rollers (not shown) inside the mill's roller bin 602 gapped at approximately one millimeter. The microbial medium crumbs 515 leaves the brewer's mill by the exit tube 608 as a coarse powder 605.

Referring to FIG. 7, the step of mixing 128 the microbial medium 605 with dextrose using a shaker box 702 is depicted. Blend the coarse microbial powder 605 with dextrose with 2 to 3 parts dextrose to 1 part microbial powder on the shaker screen 704. Use a fine dextrose product without particulates that is clean and does not fall out in a water solution. Place the dextrose and the coarse microbial powder 605 on a shaker screen 704 using a 60-mesh to 100-mesh screen to create the microbial powder blend 715. On the shaker deck 704, the ball bearings gently push the microbial dextrose mix 705 down on the screen 704. An assortment of light-weight flexible nylon balls ranging from a quarter (¼) inch diameter to a one (1) inch diameter is preferred. The microbial mixture 705 is shaken into a horizontal cement mixer 712 to ensure the resulting blend 715 stays moving.

Referring to FIG. 8, the step of diluting 130 the blended powder 715 is depicted. A tumbler 800 is used to dilute the end product achieving the final desired dilution such as 5 parts dextrose to 1part microbial powder 605. The tumbler 800 sitting on its stand 808 should have enough space inside the chamber 802 for the mixture to become airborne for allow optimal mixing. The rollers 804 rotate the tumbler 800. A motor 804 powered by a power cord 812 tumbles the end over end tumbler. A lid 806 keeps the product contained during the tumbling. After leaving the tumbler, the final product is ready to be packaged and sold.

1. A method of lyophilizing microbes in a medium, comprising: cooking a food source that includes amino acids, adding the food source to the microbes, adding a lyophilizing buffer to the microbes, lyophilizing the microbes to suspend the microbes in a medium, and powderizing the medium.

2. The method of claim 1, wherein the medium is 100 percent soluble at ambient temperatures and pressures.

3. The method of claim 1, wherein powderizing the medium comprises: turning the medium into a course powder and using a shaking mechanism to turn the coarse powder into a fine powder.

4. The method of claim 1, wherein powderizing the medium is performed at or below 75 degrees Fahrenheit or 33% humidity.

5. The method of claim 1, wherein the lyophilizing buffer comprises of sugar, wherein the sugar cane is mixed with water to form a solution between 5 percent and 10 percent inclusive of sugar after adding the buffer to the microbes.

6. The method of claim 1, wherein the cooking of the food source is performed at a temperature sufficient and time to kill contaminating microbes.
